(19) United States
(12) Patent Application Publication
(10) Pub. No.: US 2003/0129731 A1
(43) Pub. Date: Jul. 10, 2003

(54) Culture medium for detecting micro-organisms

(76) Inventor: Alain Rambach, Paris (FR)

Correspondence Address: BURNS, DOANE, SWECKER & MATHIS, L.L.P., P.O. Box 1404, Alexandria, VA, 22313-1404, US

(21) Appl. No.: 10/335,895

(22) Filed: Jan. 3, 2003

(30) Foreign Application Priority Data
Jan. 4, 2002 (FR) 02/00076

(51) Int. Cl. C12N 1/20; C12N 5/00

(52) U.S. Cl. 435/253.6; 435/404

(57) ABSTRACT

A culture medium to detect microorganisms containing carrageenan as a gelling factor, the carrageenan is preferably iota carrageenan. The culture may also comprise an agent releasing oxygen, such as nitrate oxide.

The present invention relates to a new culture medium to detect micro-organisms, containing a carrageenan, as a gelling factor.

The identification of contaminant micro-organisms is very important, in particular in the fields of food industry or the care hospital.

One can carry out this detection by many means and in particular the amplification of the specific nucleic acids of the required contaminants. However, this method requires the follow-up of a process which cannot always be made in routine.

One can also carry out detection by the growth of the micro-organisms on adequate culture media, and the analysis of the obtained colonies. For this, one can complement the culture medium with elements allowing to improve the identification of the contaminants, for example chromogenic agents, releasing chromophores precipitating in the solid medium, after hydrolysis by specific enzymes of the target micro-organisms.

However, the use of such mediums can prove to be problematic in certain cases. Indeed, the already solidified mediums have a lifespan (shelf-life) rather short and must be preserved preferably in the cold. Thus, one prefers to use dehydrated mediums, being presented in the form of powder, with a longer lifespan on the shelf, and one prepares the medium where necessary.

These dehydrated mediums contain agar as a gelling agent, which has the disadvantage of gelling only while cooling after being put in the presence of hot water. Thus, one generally autoclaves the medium, then pours it in Petri dishes and lets it take in mass. This cannot be carried out when one does not have possibility of heating water to prepare the medium.

It is thus advisable to find a means of preparing gelled culture media, even if one does not have an autoclave, or without using hot water, necessary for the dissolution and gelling of agar.

It was observed that one can manufacture a culture medium by using, as a gelling agent, a carrageenan, and that the medium takes in mass in the presence of water, even cold. The culture medium thus prepared allows the growth of the micro-organisms, as on a medium containing agar.

The carrageenans are polysaccharides resulting from the algae Chondrus crispus, and are well-known in food and cosmetic industries. They exist of three kinds, the kappa, iota, and lambda.

Thus, the present invention refers to a dehydrated culture medium containing a sufficient quantity of carrageenan to allow the formation of a gelled medium after addition of water, said carrageenan being any of the three above mentioned carrageenans, preferably the iota carrageenan, or the kappa carrageenan.

One of the ways of using the product is to add the sample to be analyzed directly on the powdered medium and to mix. The medium then gels in masse and the micro-organisms that are present grow directly within the volume created by the gelled medium (globules in the 3-dimensions medium), which can be a problem if anaerobe is too high in the medium. Thus, in order to allow a better growth of the micro-organisms in the culture medium, one can add, if necessary, an agent releasing oxygen, in particular nitrate, in particular an oxide of nitrate.

In order to reduce the risks of degradation of the product, one preferably packages it safe from ambient moisture. Thus, the invention also refers to a kit for preparing a culture medium comprising a medium according to the invention, conditioned in a container hermetic to moisture. Alternatively, the kit according to the invention comprises a medium according to the invention as well as a dessicator element making it possible to avoid the hydration of the dehydrated medium.

Generally, the invention refers to the use of a carrageenan, in particular the iota carrageenan or the kappa carrageenan, as a gelling agent in a gelled culture medium.

The invention also refers to a method of preparation of a gelled culture medium comprising the step of mixing water with a culture medium according to the invention, in particular when water is at a temperature lower than 50° C., even lower than 40° C.

The culture medium according to the invention contains the nutriments necessary to the growth of the target micro-organisms, and/or the inhibition of certain parasitic micro-organisms and is similar to the mediums known by the person skilled in the art, the agar being replaced by the carrageenan. The medium according to the invention can also contain agents for the identification of the micro-organisms, and in particular chromogenic or fluorogenic agents.

As an example, the composition of a culture medium according to the invention can be as follows (for 1 liter):

| | | |
|---|---|---|
| Iota carrageenan | 25 | g |
| Deoxycholate | 1 | g |
| KNO₃ | 1 | g |
| KH₂PO₄ | 0.66 | g |
| Na₂HPO₄ | 2.3 | g |
| NaCl | 7.5 | g |
| Peptone | 5 | g |
| Yeast extract | 2 | g |
| Magenta-glucoside | 0.05 | g |
| X-glucuronide | 0.05 | g |
| Meth-glucuronide | 0.1 | g |
| Cellobiose | 0.1 | g |

Such a medium is particularly adapted for the detection of coliforms, but it is understood that the addition of others nutriments or growth elements makes it possible to define mediums to detect other organisms.

What is claimed is:

1. A dehydrated culture medium containing a carrageenan as a gelling agent.

2. The culture medium according to claim 1, wherein said carrageenan is the iota carrageenan.

3. The culture medium of claim 1, further including an agent releasing oxygen.

4. The culture medium of claim 3, wherein said agent releasing oxygen is a nitrate oxide.

5. A kit for preparing a culture medium comprising the medium of claim 1 packaged in a container hermetic to moisture.

6. A kit for preparing a culture medium comprising the medium of claim 1, as well as a dessicator element allowing to avoid the hydration of the dehydrated medium.

7. The use of a carrageenan as a gelling agent in a gelled culture medium.

8. The use of claim 7, wherein said carrageenan is the iota carrageenan.

9. A method of preparation of a culture medium gelled including the step of mixing water with the culture medium of claim 1.
